(19) United States
(12) Patent Application Publication
(10) Pub. No.: US 2006/0040260 A1
(43) Pub. Date: Feb. 23, 2006

(54) Composition and method for monitoring in vitro conversion of full -length mammalian prion protein to amyloid form with physical properties of PRPsc

(76) Inventor: Ilia V. Baskakov, Columbia, MD (US)

Correspondence Address: INTELLECTUAL PROPERTY / TECHNOLOGY LAW, PO BOX 14329, RESEARCH TRIANGLE PARK, NC, 27709, US

(21) Appl. No.: 11/051,295

(22) Filed: Feb. 4, 2005

Related U.S. Application Data
(60) Provisional application No. 60/602,430, filed on Aug. 18, 2004.

(51) Int. Cl. C12Q 1/70; C12N 9/00

(52) U.S. Cl. 435/5; 435/183

(57) ABSTRACT

The present invention relates to an automated in vitro method for converting a prion protein into multiple forms including β-oligomer or amyloid forms while monitoring the mechanism and progress of the molecular conversion.

### CROSS-REFERENCE TO RELATED APPLICATIONS

This application claims priority from U.S. Provisional Patent Application No. 60/602,430 filed on Aug. 18, 2004 in the name of Ilia V. Baskakov for “METHOD FOR MONITORING IN VITRO CONVERSION OF FULL-LENGTH MAMMALIAN PRION PROTEIN TO AMYLOID FORM WITH PHYSICAL PROPERTIES OF PRP^{sc}.”

### BACKGROUND OF THE INVENTION

1. Field of Technology

The present invention relates to prion proteins, and more particularly, to a composition and method for converting a prion protein into multiple forms including β-oligomer and amyloid forms.

2. Description of Related Art

Several neurodegenerative maladies that can be infectious, inherited or sporadic in origin are related to the misfolding of the prion protein (PrP) (1). A central event in all three orogons of prion diseases is the conversion of the normal cellular isoform of the prion protein, PrP^{C}, into the abnormal pathological isoform, PrP^{Sc}. This conversion involves a substantial conformational change: PrP^{C} is a proteinase K (PK)-sensitive α-helical monomer, whereas PrP^{Sc} is an assembled multimer characterized by enhanced resistance toward PK-digestion and a higher content of β-structure (2; 3). To explain the infectious form of prion diseases, the “protein only” hypothesis postulates that PrP^{Sc} acts as a transmissible agent and that it self-propagates its pathological conformation in an autocatalytic manner using PrP^{C} as a substrate (4).

Substantial effort has been dedicated to the development of a cell-free conversion system for reconstitution of the infectious PrP^{Sc} from recombinant PrP in vitro (5; 6). To study the conversion in vitro, truncated rPrP encompassing residues 90-231 has been widely used (7-12). rPrP 90-231 corresponds to the protease K-resistant core of the PrP^{Sc} referred to as PrP 27-30, which is generated by cleavage of the N-terminus around amino acid residue 90 (13). Because PrP 27-30 is capable of transmitting prion disease (14) and because transgenic mice expressing only PrP 90-231 but not the full length PrP^{C} support prion propagation (15), the N-terminus is believed to be unnecessary for the development of prion disease.

While the N-terminus of PrP is not important for transmission of prions, this region seems to be involved in the cellular function of PrP^{C}. The N-terminal domain contains an octarepeat region (residues 60-90) which displays high affinity for binding of Cu²⁺ ions (16; 17). This domain is highly flexible in the absence of Cu²⁺ (18; 19). However, it adopts a unique structure upon binding four Cu²⁺ ions (20; 21). In addition, a fifth Cu²⁺ binding site was identified between residues 90 and 96 adjacent to the octarepeat motif (20; 22). The N-terminal domain was also shown to bind different classes of macromolecules, including sulfated glycans and RNA (23-25), which stimulated PrP^{Sc}-dependent cell-free conversion of PrP^{C} into the proteinase K-resistant PrP isoform (26-28). Because of its high affinity for Cu²⁺ and its ability to bind cellular macromolecules, the N-terminal region may affect the pathways of misfolding and influence the conformational diversity of abnormal β-sheet rich isoforms generated in vivo. Thus, the length of PK-resistant fragments generated upon treatment of PrP^{Sc} were Cu²⁺-dependent (29). It is reasonable to speculate, that the N-terminal region, although not essential for infectivity, may in fact substantially impact the conformational diversity of PrP^{Sc} strains and subtypes and, therefore, assist in the cell-free conversion of recombinant PrP into the infectious isoform. However, due to a number of technical difficulties, oxidized full-length PrP has never been converted into the amyloid form.

Thus, it would be advantageous to develop a system and method for converting a full-length prion protein into an amyloid form for studying the molecular mechanism of prion diseases

### SUMMARY OF THE INVENTION

The current studies provide the first demonstration that full-length recombinant PrP with an intact S—S bond can be folded into amyloid conformation in vitro. This conversion mimics a transmission barrier of prion replication observed in vivo and can be achieved at physiological concentrations of PrP (1 uM). Furthermore, the proteinase K (PK)-resistant C-terminal core of the amyloid form maintains a β-sheet rich conformation and preserves high seeding activity.

In one aspect, the present invention provides for an in vitro method for converting a full-length recombinant prion protein into an amyloid form thereby providing a model for studying the molecular mechanism of prion diseases.

In another aspect the present invention provides for an in vitro method for converting a prion protein to an amyloid form, the method comprising:
a) providing a conversion solution comprising guanidine hydrochloride (GdnHCl);
b) adding a recombinant full-length prion protein to the conversion solution;
c) maintaining the pH in the solution in a range from about 5.5 to about 6.5;
d) exposing the recombinant prion protein to the solution under essentially continuance shaking for a sufficient time to form an amyloid structure.

In yet another aspect, the present invention provides for an in vitro method for converting a prion protein to a β-oligomer form, the method comprising:
a) providing a conversion solution comprising guanidine hydrochloride (GdnHCl);
b) adding a recombinant full-length prion protein to the conversion solution;
c) maintaining the pH in the solution in a range from about 3.0 to about 4.0;
d) exposing the recombinant prion protein to the solution for a sufficient time to form a β-oligomer form.

In another aspect, the present invention provides for an automated method of monitoring conversion kinetics of the conversion of a full-length prior protein or fragments thereof, the method comprising:
a) providing a conversion solution comprising guanidine hydrochloride (GdnHCl) and Thioflavin T (ThT);
b) adding a full-length prion protein or fragment thereof to the conversion solution;
c) maintaining the pH in the solution in a range from about 5.5 to about 6.5;
d) exposing the prion protein to the solution under essentially continuance motion; and
e) monitoring the conversion kinetics to an amyloid structure by measuring the fluorescence intensity corresponding to the conversion.

A still further aspect of the present invention provides for an automated method of monitoring conversion kinetics of the conversion of a full-length prior protein or fragments thereof, the method comprising:
a) providing a conversion solution comprising guanidine hydrochloride (GdnHCl) and Thioflavin T (ThT);
b) adding a full-length prion protein or fragment thereof to the conversion solution;
c) maintaining the pH in the solution in a range from about 3.0 to about 4.0;
d) exposing the prion protein to the solution under essentially continuance motion; and
e) monitoring the conversion kinetics in forming a β-oligomer by measuring the fluorescence intensity corresponding to the conversion.

In another aspect the present invention provides for an automated method for determining test compounds that inhibit or reduce the conversion of a full-length prior protein or fragments thereof into an amyloid form, the method comprising:
a) providing a conversion solution comprising guanidine hydrochloride (GdnHCI) and Thioflavin T (ThT);
b) adding a full-length prion protein or fragment thereof to the conversion solution;
c) maintaining the pH in the solution in a range from about 5.5 to about 7.0;
d) exposing the prion protein to the solution under essentially continuance motion;
e) introducing the test compound; and
f) monitoring the conversion kinetics relative to a control sample without the test compound by measuring the fluorescence intensity corresponding to the conversion.

Another aspect of the present invention provides for an automated method for determining test compounds that inhibit or reduce the conversion of a full-length prior protein or fragments thereof into a β-oligomer form, the method comprising:
a) providing a conversion solution comprising guanidine hydrochloride (GdnHCl) and Thioflavin T (ThT);
b) adding a full-length prion protein with an intact S—S bond or fragment thereof to the conversion solution;
c) maintaining the pH in the solution in a range from about 3.0 to about 4.0;
d) exposing the prion protein to the solution under essentially continuance motion;
e) introducing the test compound; and
f) monitoring the conversion kinetics relative to a control sample without the test compound by measuring the fluorescence intensity corresponding to the conversion.

A further aspect of the present invention relates to a kit for determining test compounds that inhibit or reduce the conversion of a full-length prior protein or fragments thereof into a β-oligomer or amyloid form, the kit comprising:
a) a conversion solution comprising guanidine hydrochloride (GdnHCl) and Thioflavin T (ThT);
b) a pH altering compound for maintaining the conversion in a range from about 3.0 to about 7.0, wherein a full-length prion protein and test compound are added to the conversion solution and monitoring conditions to determine if the test compound inhibits or reduces conversion.

### BRIEF DESCRIPTION OF THE FIGURES

FIGS. 1 A, B, C, and D show the in vitro conversion of rPrP into the β-oligomer and to the amyloid form. (A) Size-exclusion chromatography profiles of original α-rPrP (22 uM) (- · +19 -) and upon incubation of α-rPrP at 37° C. in 1 M GdnHCl, 3 M urea, 150 mM NaCl pH 3.7 for 1 h ( [symbol] ), 2 h ( [symbol] ), 4 h (- - - -), 10 h (- - -), and 27 h ( [symbol] ). Profiles of original α-rPrP showed that 14% of protein had already converted to the oligomeric form during preparation of the stock solution of rPrP (130 uM) in 6 M GdnHCl. The elution time of the oligomeric and the monomeric species were 7.1 min and 11.2 min, respectively. (B) Far UV CD spectra of rPrP (11 uM) predominantly composed of the α-rPrP (85% of α-rPrP and 15% of the β-oligomer as assessed by size-exclusion chromatography)—solid line, and the oligomeric form (80% of the β-oligomer and 20% of α-rPrP)—dashed line. Samples of rPrP were prepared as described in Materials and Methods and dialyzed against 10 mM Na-acetate buffer pH 5.0 before measurements. (C) The kinetics of rPrP (22 uM) conversion into the β-oligomer monitored by size-exclusion chromatography as a function of pH: 3.7 (●), 5.5 (◯), and 6.8 (▾). (D) The kinetics of rPrP (22 uM) conversion into the amyloid form monitored by ThT-binding assay as a function of pH: pH 3.7 (●), pH 5.5 (◯), and pH 6.8 (▾). Formation of both the β-oligomer and the amyloid fibrils was carried out at 37° C. in 1 M GdnHCl, 3 M urea, 150 mM NaCl in either 20 mM Na-acetate buffer (for pH 3.7 or 5.5), or 20 mM potassium-phosphate buffer (for pH 6.8). To form amyloid fibrils the reaction mixtures were incubated with continuous shaking at 600 RPM, while conversion to the β-oligomer was carried out under identical solvent conditions, but did not require shaking.

FIGS. 2 A, B, C and D show that the P-oligomer and the amyloid form have distinct conformational properties. (A) ThT fluorescence measured in the presence the β-oligomer (◯), the amyloid form (●), and in the absence of rPrP (▾). Concentration of rPrP was 1 uM in both samples. The slight decline of ThT-fluorescence observed above 30 uM is due to self-absorbance effect. (B) FTIR spectra of rPrP in predominantly α-monomeric form (85% of α-rPrP and 15% of the β-oligomer as assessed by size-exclusion chromatography, solid line), predominantly β-oligomeric form (80% of the β-oligomer and 20% of α-rPrP, dotted line), or the amyloid form (dashed line). Preparation of rPrP isoforms is described in Materials and Methods. (C) Electron micrographs of the β-oligomer (panel 1), the amyloid fibrils (panel 2), and gallery of fibrils: a single filament (panel 3); ‘unzipped’ fibrils (panels 4, 5); a flat ribbon-like fibril composed of two filaments (panel 6). (D) Limited PK digestion of the β-oligomer (panel 1) and the amyloid form (panels 2-4) followed by Western blot with Fabs P (epitope 96-105, panels 1,2), with Fabs R1 (epitope 225-230, panel 3), and anti-prion serum Ab-79-97 (epitope 79-97, panel 4). Both isoforms of rPrP (0.2 mg/ml) were treated with PK for 1 h at 37° C. at the following PK/rPrP ratios: 1:10,000 (lanes 2), 1:5,000 (lanes 3), 1:1,000 (lanes 4), 1:500 (lanes 5), 1:100 (lanes 6), and 1:50 (lanes 7); no PK (lanes 1). Apparent molecular masses of PK-resistant fragments are given in kDa.

FIGS. 3 A and B show that in vitro conversion into the amyloid form mimics a transmission barrier. (A) The kinetics of amyloid formation for rPrP 106 (5 uM) seeded with 2% (green and yellow circles, duplicate runs) and 0.2% (light blue and dark blue circles, duplicate runs) of fibrillar rPrP 106, with 2% of fibrillar full-length rPrP (magenta and pink circles, duplicate runs), and without seeding (orange and brown circles, duplicate runs). The amyloid fibrils of both rPrP106 and rPrP used for seeding were produced using the manual format by incubating the reaction mixture of rPrP106 (22 uM) or rPrP (22 uM), respectively, at 37° C. in 1 M GdnHCl, 3 M urea, 150 mM NaCl, and 20 mM potassium-phosphate buffer (pH 6.8) in the reaction volume 0.6 ml as described in Material and Methods. (B) The kinetics of amyloid formation for full-length rPrP (2 uM) seeded with 2% of fibrillar full-length rPrP (orange and brown circles, duplicate runs), with 2% of fibrillar rPrP 106 (light blue and dark blue circles, duplicate runs), and without seeding (green and yellow circles, duplicate runs). The conversion reactions presented in panels A and B were carried out in a 96-weel plate at 37° C. in 1 M GdnHCl, 3 M urea, 150 mM NaCl, and 20 mM potassium-phosphate buffer (pH 6.8) using the automated format as described in Material and Methods. The amounts of rPrP 106 and full-length rPrP seeds are calculated based on molar equivalents.

FIGS. 4 A and B show that FTIR spectra reveal remarkable stability of the β-structures of the amyloid form toward thermal denaturation and PK-digestion. FTIR spectra (A) and their second derivatives (B) of the amyloid form (0.5 mg/ml) recorded in the time course of thermal denaturation (B, top panel) and renaturation (B, bottom panel) at the following temperatures: 20° C. (-··-··-··-), 40° C. ( [symbol] ), 60° C. (---------), and 80° C. (------). FTIR spectra (C) and their second derivatives (D) of the amyloid form without PK (-··-··-··-), and treated with PK at 37° C. for 20 min ( [symbol] ), 40 min ( [symbol] ), 1 h (---------), 2.5 h (------), and 4 h ( [symbol] ) at PK/rPrP ratio 1:100.

FIGS. 5 A and B show (A) Electron micrographs of negatively stained intact amyloid fibrils taken at 20,000× Limited PK-digestion induces lateral aggregation of the amyloid fibrils. magnification (panel 1) and the amyloid fibrils treated with PK at 37° C. for 1 h at PK/rPrP ratio of 1:500 taken at 20,000× (panels 2, 3), 4,000× (panel 4) and 2,000× (panels 5, 6) magnifications. (B) Fluorescence microscopy of the amyloid fibrils taken before addition of PK (panel 1, amyloid fibrils are attached to a surface of cover slip), and after incubation with PK for 10 min (panel 2, fibrils get detached from the surface and aggregate in solution) and 30 min (panel 3, fibrils form large clumps). PK/rPrP ratio is 1:500.

FIGS. 6 A and B show that harsh PK-digestion of the amyloid fibrils induces their fragmentation. The amyloid fibrils were incubated with PK for 1 h at 37° C. at PK/rPrP ratio of 1:50. (A) Electron micrographs of negatively stained amyloid fibrils treated with PK illustrate numerous bending and fragmentation. (B) Gallery of fibrils: untwisted fibrils composed of two ribbons (2); twisted fibrils composed of two ribbons (3,4); twisted fibrils composed of more than two ribbons (5, 6); fibrils displaying ‘unzipped’ ribbons (2-4) or ‘unzipping’ of a single filament (5), fibrils with bending that occurs across whole fibrillar diameter (11) or across diameter of a single ribbon (7); fragmentation of fibrils (7-10, 12). Arrows show points of bending or fragmentations. Example of typical PK-nontreated fibril is shown for comparison on panel 1. The scale bars=50 nm.

FIGS. 7 A, B and C show Epifluorescence microscopy imaging of intact amyloid fibrils (A) and fibrils treated with PK (B). The scale bars=2 um. (C) Fluorescence images of single fibrils: typical intact fibril (panel 1), fibrils after treatment with PK (panels 2, 3). The amyloid fibrils were incubated with PK for 1 h at 37° C. at PK/rPrP ratio of 1:50.

FIG. 8 shows that the PK-resistant core of the amyloid form displays seeding activity. The kinetics of amyloid formation for rPrP (1 uM) seeded with 1% of amyloid form pretreated with PK (light blue and dark blue circles, duplicate runs), and with 1% of intact amyloid form (green and yellow circles, duplicate runs). The orange and brown circles represent the kinetics in non-seeded reactions. The amyloid form used for seeding was incubated with PK at 37° C. for 1 h at the PK/rPrP ratio of 1:50. The in vitro conversion was carried out in 96-weel plate at 37° C. in 1 M GdnHCl, 3 M urea, 150 mM NaCl, and 20 mM potassium-phosphate buffer (pH 6.8) using the automated format as described in Materials and Methods.

FIG. 9 shows a schematic diagram illustrating the complexity of in vitro conversion pathways. The β-oligomer is formed at acidic pH and the amyloid ribbons are generated at neutral pH. Upon PK-digestion the amyloid ribbons either aggregate into large clumps, or assemble into fibrils composed of 2 or more ribbons. Amyloid fibrils break into short fragments creating new active centers for propagation.

FIG. 10 shows the nucleotide and amino acid sequence for mammalian prion protein discussed herein.

### DETAILED DESCRIPTION OF THE INVENTION

As defined herein, “prion protein” may be a “normal” prion protein, also referred to as a “sensitive” prion protein, and may be designated “PrPc” protein. The prion protein may also be an infectious form of the protein, also called a “resistant” or “scrapie” form, and may be designated “PrP^{SC}” protein. Also included in the definition of prion protein are variants of the sensitive and resistant forms of the prion protein. Prion protein variants herein include all isoforms of both the sensitive and resistant forms and all isolates or strains of prion protein. The isolates or strains may vary by structure or conformation, or by characteristic incubation times of the disease, disease length and pathology. The amino acid sequences of the variants may also vary by one or more amino acids.

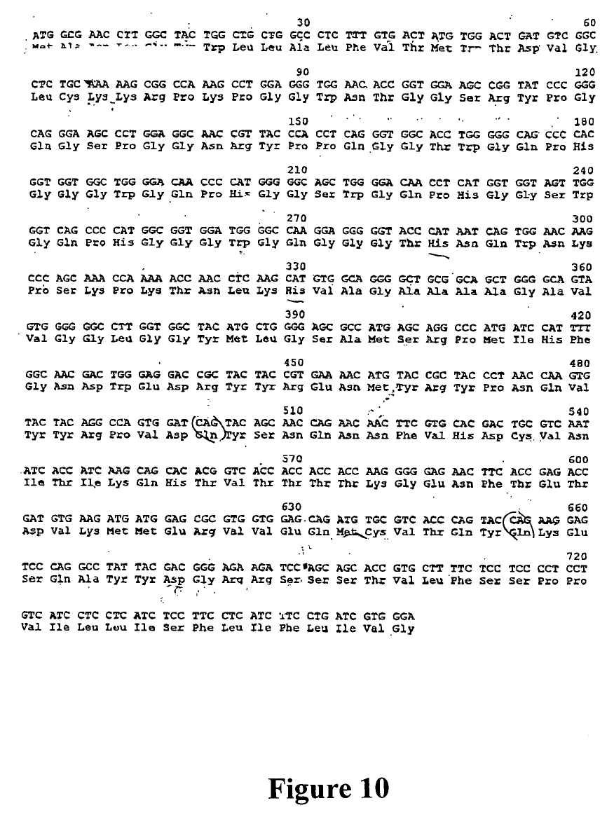

The ‘protein only’ hypothesis postulates that the infectious agent of prion diseases, PrP^{Sc}, is composed of the prion protein (PrP) converted into an amyloid-specific conformation. However, cell-free conversion of the full-length PrP into the amyloid conformation has not yet been achieved. In an effort to understand the mechanism of PrP^{Sc} formation, the present invention provides for a cell-free conversion system using recombinant mouse full-length PrP (FIG. 10) with an intact disulfide bond (rPrP). The present invention demonstrates that rPrP will convert into the β-sheet rich oligomeric form under highly acidic pH (<5.5) and at high concentrations, while under slightly acidic or neutral pH (>5.5) it assembles into the amyloid form. As judged from electron microscopy, the amyloid form had a ribbon-like assembly composed of two non-twisted filaments. In contrast to the formation of the β-oligomer, the conversion to the amyloid occurred at concentrations close to physiological and displayed key features of an autocatalytic process. Moreover, using a shortened rPrP consisting of 106 residues (rPrP 106, deletions: Δ23-88 and Δ141-176), we showed that the in vitro conversion mimicked a transmission barrier observed in vivo. Furthermore, the amyloid form displayed a remarkable resistance to proteinase K (PK) and produced a PK-resistant core identical to that of PrP^{Sc}. FTIR analyses showed that the β-sheet rich core of the amyloid form remained intact upon PK-digestion and accounted for the extremely high thermal stability. Electron and real-time fluorescent microscopy revealed that proteolytic digestion induces either aggregation of the amyloid ribbons into large clumps or further assembly into fibrils composed of several ribbons. Fibrils composed of ribbons exhibited high fragility and a tendency to fragment into short pieces. Remarkably, the amyloid form treated with PK preserved high seeding activity. The present invention shows that the amyloid form, which recapitulates key physical properties of PrP^{Sc}, can be achieved in vitro in the absence of cellular factors or a PrP^{Sc} template.

It is understood that modification that do not substantially affect the activity of the various embodiments of this invention are also included within the definition of the invention provided herein. Accordingly, the following examples are intended to illustrate but not limit the present invention.

### EXAMPLES

Material and Methods

Protein Expression and Purification.

Mouse PrP 23-231 DNA (FIG. 10) was PCR amplified from pcDNA3 plasmids containing the full length PrP gene, inserted into pET101/D-TOPO vector (Invitrogen) and transformed into Top10 cells (Invitrogen). The transformants were tested by PCR amplification, the DNAs from the positive clones were checked by DNA sequencing and retransformed into BL21 (DE3) Star cells (Invitrogen). For expression, transformants were inoculated into 10 ml of LB/carbenicillin medium (0.1 mg/ml carbenicillin) and were grown at 37° C. for 3.5 h. The entire culture was inoculated into 100 ml of LB/carbenicillin medium and grown overnight (˜16 h). 5% of the overnight culture was inoculated into TB medium (300 ml) supplemented with carbenicillin (0.1 mg/ml) and grown at 37° C. until the A_{600 nm} reached 0.6. Expression was induced by addition of isopropyl-β-D-thiogalactopyranoside (Sigma) to a final concentration of 1 mM and the cultures were grown for an additional 5 h. Cells were harvested by centrifugation, resuspended in lysis buffer (50 mM Tris, 1 mM EDTA, 100 mM NaCl, pH 8; 9 ml per gram of pellet), followed by the addition of lysozyme (200 ug/ml) and PMSF (20 ug/ml) with subsequent incubation on ice for 20-40 min. Deoxycholic acid (1 mg/ml) was added followed by incubation on ice for 20-30 min, subsequent addition of DNAse (10 ug/ml), and a final incubation for 30-45 min. The lysate was centrifuged at 20,000×g for 20 min. The resulting pellet was dissolved in IMAC buffer A (5 ml per gram of pellet, 0.1 M Na₂HPO₄, 10 mM Tris, 8 M Urea, 10 mM β-mercaptoethanol, pH 8;), incubated for 2 h at room temperature, and centrifuged at 20,000×g for 15 min to remove insoluble material. The solubilized inclusion bodies were incubated with NTA Fast Flow Sepharose resin (Amersham Biosciences, Sweden) pre-charged with Ni-ions at room temperature for 1 h in a top-bottom mixer. The NTA column was washed with five volumes of IMAC buffer A, followed by elution of rPrP in IMAC buffer B (0.1 M Na₂HPO₄, 10 mM Tris, 8 M Urea, 10 mM β-mercaptoethanol, pH 4.5). Fractions containing rPrP were diluted to a final protein concentration of 0.5 mg/ml using 9 M urea in 0.1 M Tris buffer pH 8.0 and dialyzed against 9 M urea in 0.1 M Tris buffer pH 8.0 to eliminate β-mercaptoethanol. The dialyzed solution was diluted 3-fold with buffer I (0.1% trifluoroacetic acid/H₂O), loaded on a 25 mm×25 cm C4 HPLC column (Vydac), and eluted using a gradient of buffer II (0.1% trifluoroacetic acid/acetonitrile). Fractions containing rPrP were eluted in 40% acetonitrile and lyophilized. The purity of final rPrP preparation was confirmed by SDS-PAGE followed by silver staining and electrospray mass spectrometry to be a single species with an intact disulfide bond. 10 mg of 99.5+% pure rPrP were obtained per liter of culture. Recombinant mouse PrP of 106 amino acid residues was purified as described before (46).

In vitro conversion of rPrP to the α-monomeric form, to the the β-oligomer and to the amyloid fibrils.

To convert rPrP to the α-rPrP, a stock solution of rPrP (130 uM) in 6 M GdnHCl was diluted to the final protein concentration of 22 uM in 20 mM Na-acetate buffer pH 5.0 at room temperature and dialyzed against 10 mM Na-acetate buffer pH 5.0.

To form amyloid fibrils two different formats were used, that being both manual and automated. In the manual format, a stock solution of rPrP (130 uM) in 6 M GdnHCl was diluted to the final protein concentration of 22 uM and incubated at 37° C. in 1 M GdnHCl, 3 M urea, 150 mM NaCl with continuous shaking from about 400 to 700 RPM and preferably at 600 RPM using a Delfia plate shaker (Wallac) in conical plastic tubes (Eppendorf) in a reaction volume >0.4 ml. The conversion reactions at pH 3.7 and 5.5 were carried out in 20 mM Na-acetate buffer and at pH 6.8 in 20 mM potassium-phosphate buffer. The kinetics of fibril formation were monitored using a ThT-binding assay. Aliquots were manually withdrawn during the time course of incubation at 37° C. were diluted into 5 mM Na-acetate buffer (pH 5.5) to a final concentration of rPrP of 0.3 uM, then ThT (Molecular Probes, Eugene, Oreg.) was added to a final concentration of 10 uM. Six emission spectra (from 460 to 520 nm) were recorded for each sample in 0.4 cm rectangular cuvettes with excitation at 445 nm on a FluoroMax-3 fluorimeter (Jobin Yvon, Edison, N.J.), both excitation and emission slits were 4 nm. Spectra were averaged and the fluorescence intensity at emission maximum (482 nm) was determined.

Conversion to the amyloid fibrils in the automated format was carried out using the same solvent conditions as those used in the manual format but in the reaction volume of 0.2 ml in 96-well plates and importantly in the presence of ThT (10 uM). The preliminary studies using the manual format demonstrated that ThT can be included in the conversion solution and surprisingly the presence of 10 uM ThT in the reaction mixture did not interfere with the kinetics of amyloid formation (data not shown). Advantageously, the conversion could be monitored almost immediately. The 96-well plates were covered by ELAS septum sheets (Spike International), incubated at 37° C. upon continuous shaking at 900 RPM in Fluoroskan Ascent CF microplate reader (ThermoLabsystems) and the kinetics was monitored by bottom reading of fluorescence intensity every few minutes, understanding that a measurement can be taken every few second to 6 minutes using 444 nm excitation and 485 nm emission filters.

The conversion to the β-oligomer was carried out under identical solvent conditions as the formation of the amyloid fibrils, but did not require shaking. To obtain a maximal yeild of the β-oligomer for FTIR and CD experiments, the conversion reactions were carried out at pH 3.7 for 48 hours followed by dialysis against 10 mM Na-acetate buffer pH 5.0. The kinetics of conversion to the β-oligomer was monitored by HPLC size exclusion chromatography at 23° C. with a flow rate of 0.3 ml/min using a 4.6 mm×30 cm TSK Super SW 3000HPLC column (Tosoh Corporation, Tokyo, Japan) in a running buffer of 20 mM Na-acetate (pH 3.7), 0.2 M NaCl, and 1 M urea.

Proteinase K Digestion and Western Blot.

The β-oligomer and the amyloid fibrils of rPrP (0.2 mg/ml) were treated with PK at 37° C. for 1 h in 0.1 M Tris-HCl buffer (pH 7.2). Digestion was stopped by quenching with PMSF (2 mM), followed by addition of 8 M urea, to a final concentration of 3 M, and 4× sample buffer. Samples were heated at 95° C. for 15 min and analyzed by 12% NuPage SDS-PAGE (Invitrogen). For Western blot experiments, proteins were electroblotted onto Immobilon P PVDF membrane (Millipore), incubated with anti-PrP Fabs (0.2 μg/ml) or with anti-prion serum Ab-79-97 (1:10,000 dilution, EMD Biosciences, San Diego) followed by incubation with goat anti-human IgG F(ab′)2 fragment or anti-goat IgG conjugated with HRP, respectively, and detected using the ECL system (Pierce).

Anti-prion serum Ab-79-97 reacts with epitope including amino acid residues 79-97 (47).

Electron Microscopy.

Negative staining was performed on carbon-coated 100-mesh grids coated with 0.01% of poly-L-lysine solution prior to staining. The samples were adsorbed for 30 s, washed with 0.1 M and 0.01 M Na-acetate for 5 s each, stained with freshly filtered 2% uranyl acetate for 30 s, dried and then viewed in a Zeiss EM 10 CA electron microscope.

CD and FTIR Spectroscopy.

CD spectra of rPrP (0.25 mg/ml) were recorded in 10 mM Na-acetate buffer pH 5.0 in a 0.1-cm cuvette with a J-810 CD spectrometer (Jasco, Easton, Md.), scanning at 20 nm/min, with a bandwidth of 1 nm and data spacing of 0.5 nm. Each spectrum represents the average of three individual scans after subtracting the background spectra.

FTIR spectra were measured with a Bruker Tensor 27 FTIR instrument (Bruker Optics, Billerica, Mass.) equipped with a MCT detector cooled with liquid nitrogen. Three isoforms of rPrP (the α-monomer, the β-oligomer and the amyloid fibrils) were dialyzed against 10 mM Na-acetate buffer pH 5.0, and 10 ul of each isoform (0.5 mg/ml) were loaded into BioATRcell II. 128 scans at 2 cm⁻¹ resolution were collected for each sample under constant purging with nitrogen, corrected for water vapor and background spectra of water were subtracted. For thermal denaturation assays, the solution was heated in the BioATRcell from 20° C. to 80° C. by increasing the temperature in 10 deg. C. increments over 10 min each, equilibrated at 80° C. for 5 min, and then cooled back to 20° C. in 10 deg. C. decrements over 15 min each.

Epifluorescence Microscopy.

Epifluorescence microscopy experiments were carried out on an inverted microscope (Nikon Eclipse TE2000-U) with illumination system X-Cite 120 (EXFO Photonics Solutions Inc.) connected through fiber-optics using a 1.3 aperture Plan Fluor 100×NA objective. The emission was isolated from Rayleigh and Raman-shifted light by a combination of filters: an excitation filter 485DF22, a beam splitter 505DRLPO2, and an emission filter 510LP (Omega Optical, Inc.). Digital images were acquired using a cooled 12-bit CoolSnap HQ CCD camera (Photometrics). Prior to imaging fibrils were diluted to a final concentration of rPrP equivalent to 0.1 uM and stained with ThT (10 uM) for 3 min.

Formation of the β-Oligomeric Form Versus the Amyloid Form.

The present inventor demonstrated that rPrP 90-231 proteins (human, mouse, or hamster) adopt two abnormal β-sheet rich isoforms in vitro, the β-oligomer and the amyloid form (30; 31). Under acidic pH, rPrP 90-231 (truncated) assembles into the β-oligomer (7), whereas the conversion into the amyloid form occurs under neutral and slightly acidic pH (31). Although the truncated version behaved a specific way, there was a question as to whether the full-length rPrP with an intact S-S bond would follow the same folding behavior. As such, the present inventor analyzed the kinetics of conversion of rPrP into an oligomer and to amyloid fibrils at different pH values.

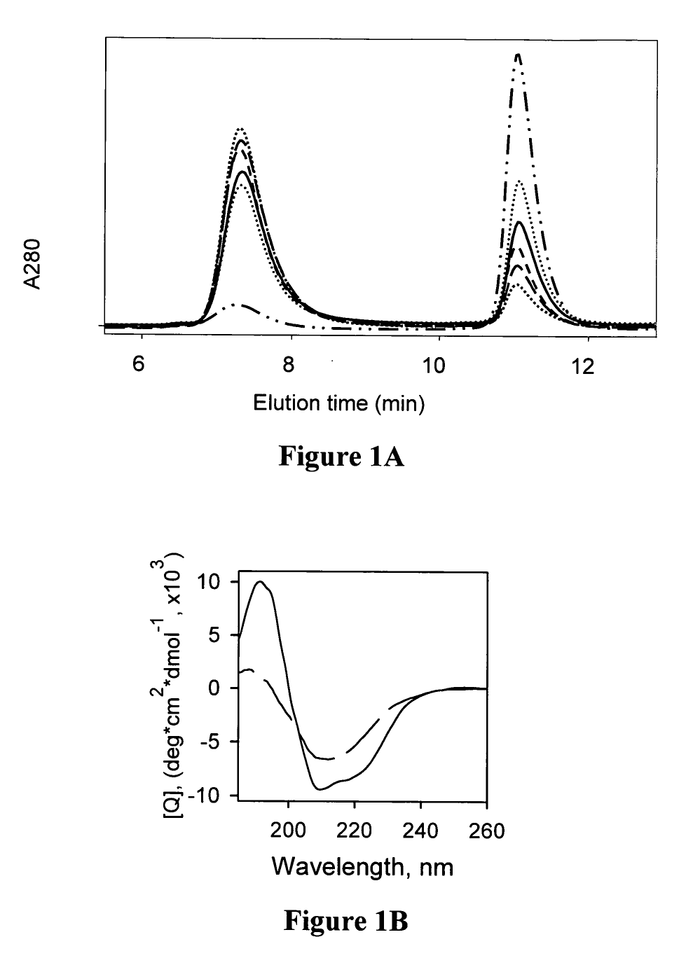

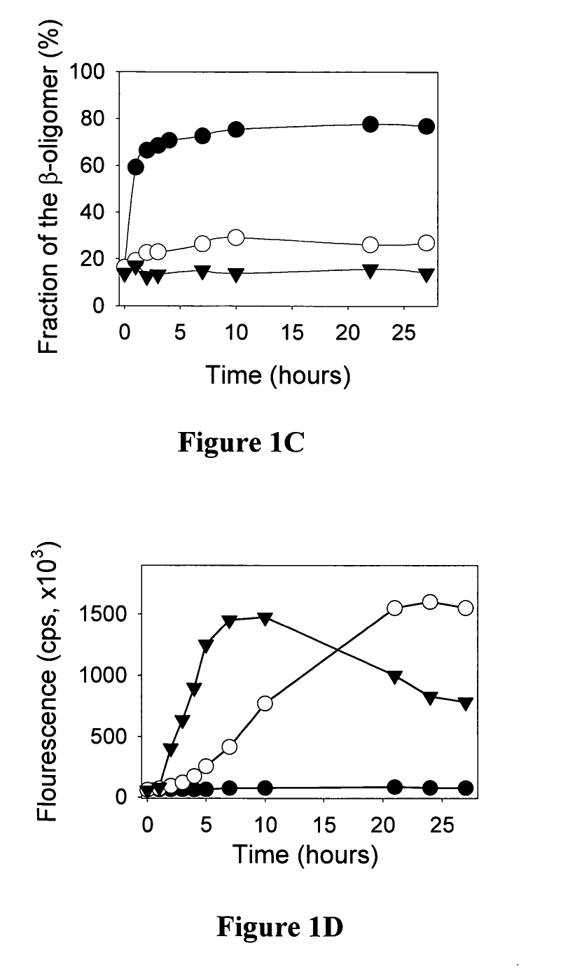

Monomeric rPrP quickly assembled into oligomeric species when incubated at pH 3.7, as monitored by size-exclusion chromatography (FIG. 1A). Because the oligomeric form had a predominant β-sheet conformation as determined by circular dichroism (CD) (FIG. 1B), it will be referred to as the β-oligomer. To analyze the kinetics of β-oligomer formation at different pH values, size exclusion chromatography was used which allows quantitative monitoring of the fractionation of the β-oligomers and monomers. The yield and rate of the β-oligomer formation were pH-dependent, where acidic pH favored the oligomerization. As shown in FIG. 1C, the kinetics of rPrP (22 uM) conversion into the β-oligomer occurred significantly at a pH of pH: 3.7 (●). As the pH increased to the neutral range there was a marked reduction in the rate of the β-oligomer formation. After 10 h of incubation at pH 3.7, 75% of rPrP was found in the β-oligomeric form, whereas only 14% of rPrP was detected in the β-oligomeric form at pH 6.8. It is not unusual for a small amount of β-oligomer to normally form during preparation of concentrated stock solution of rPrP. However, no further conversion can be detected for up to 30 h of incubation at pH 6.8 (FIG. 1C). This result was similar to the folding behavior of rPrP 90-231 which has a tendency to form minor amounts of the β-oligomeric species upon preparation of concentrated stock solution or upon refolding of rPrP 90-231 into the β-helical monomer (32).

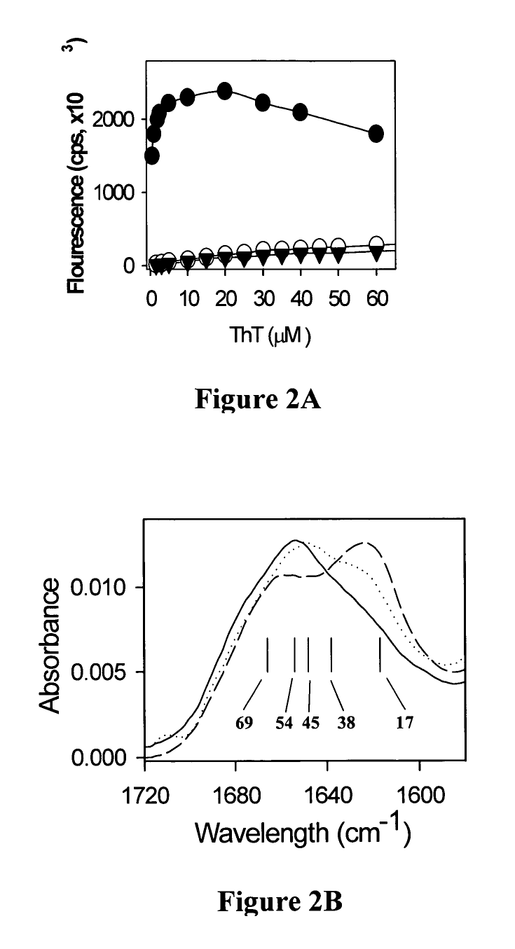

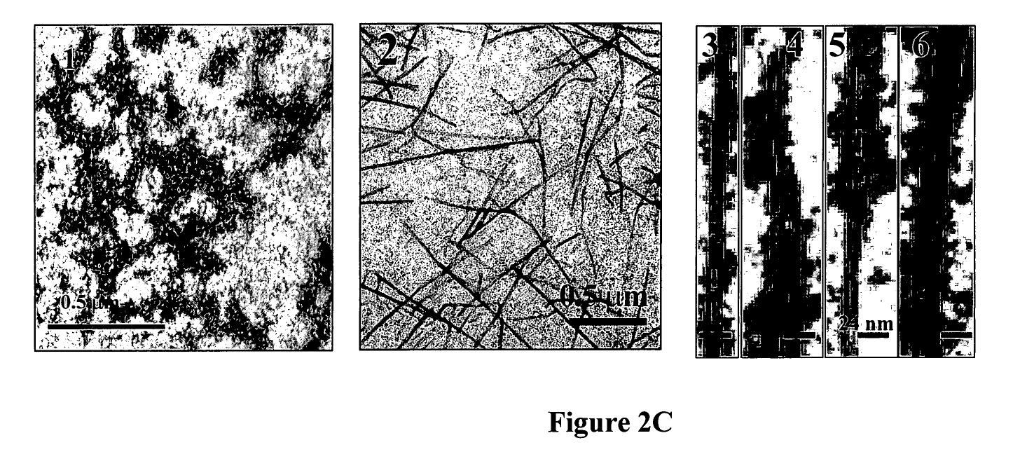

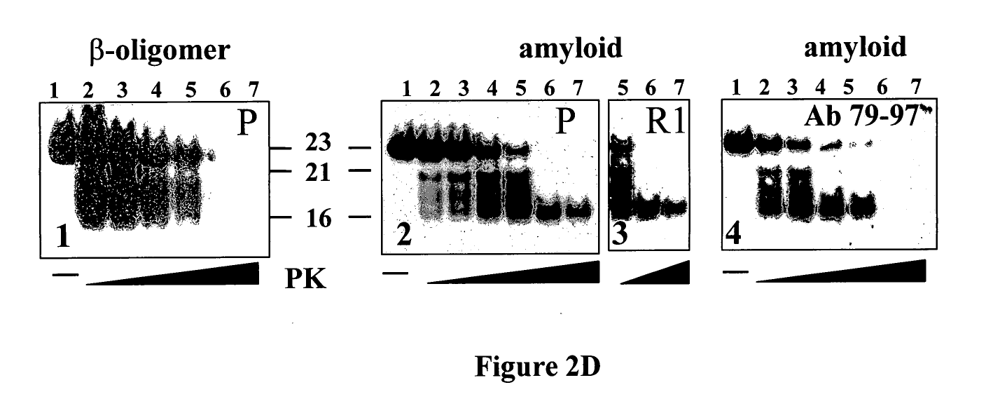

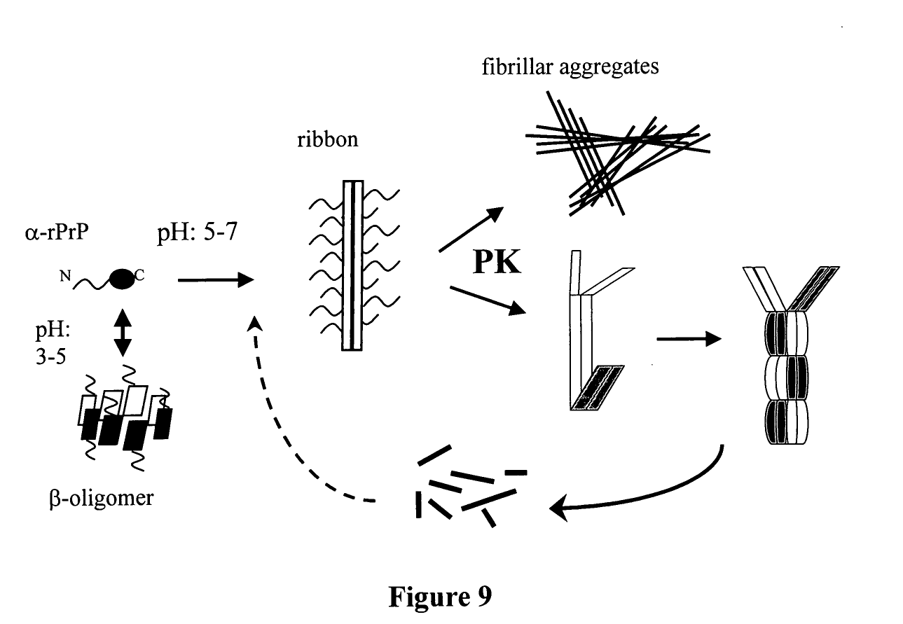

To monitor the kinetics of the amyloid formation we used a Thioflavin T (ThT)-binding assay (FIG. 1D). Both isoforms, the P-oligomer and the amyloid fibrils, bind ThT. However, the binding capacity of the amyloid fibrils is 50-100 fold higher than the capacity of an equivalent amount of the β-oligomer (FIG. 2A). The amyloid formation was carried out under solution conditions identical to that used for the formation of the β-oligomer (37° C., 1M GdnHCl, 3 M urea, 150 mM NaCl), but required continuous shaking in a range from about 400 to 700 RPM, and preferably at 600 RPM. Typical kinetics of amyloid formation displayed a lag-phase followed by rapid accumulation of fibrils. The pH-dependence of this process was inverse to that measured for the β-oligomer (FIG. 1, compare panels C and D). The shortest lag phase and the most rapid production of the amyloid were observed at pH 6.8, whereas no fibrils were found at pH 3.7 after at least 28 hours of incubation (FIG. 1D). It was found that the full length prion with an intact S—S bond formed a β-oligomer at acidic pH, while conversion to the amyloid fibrils occurs at near neutral and slightly acidic pH (FIG. 9).

Amyloid and β-Oligomer Have Distinct Conformational Properties.

As noted above, the ThT-binding capacity of the amyloid fibrils substantially exceeds that of the β-oligomer (FIG. 2A). Therefore, the ThT-binding assay offers a rapid procedure for distinguishing the two abnormal isoforms. To gain an additional insight into the structural changes that accompanied conversion of rPrP into the abnormal isoforms we used FTIR, electron microscopy, and limited PK-digestion. The FTIR spectrum acquired for α-helical monomeric form of rPrP (α-rPrP, the conversion to the α-rPrP is described in Materials and Methods) was dominated by strong absorbance at 1654 cm⁻¹ and 1645 cm⁻¹ corresponding to α-helices and random coil, respectively (FIG. 2B). Upon conversion to the β-oligomer an increased intensity of bands between 1638 cm⁻¹ and 1617 cm⁻¹ was observed, an indication of β-strand-containing structures with intra- and intermolecular hydrogen bonds. The FTIR spectrum of the amyloid form was remarkably different from that of the β-oligomer but resembled that of PrP 27-30 (33; 34). The amyloid showed substantial decrease in intensity of the bands corresponding to α-helices and random coil and an increased intensity of the band at 1617 cm⁻¹, an indication of β-structure with strong intermolecular hydrogen bonds.

Electron microscopy of the β-oligomers displayed a relatively homogeneous population of spherical particles (FIG. 2C, panel 1). In contrast, rPrP converted into the amyloid form showed long fibrils (FIG. 2C, panel 2). A close examination revealed that most fibrils had a ribbon-like assembly composed of two laterally aligned non-twisted flat filaments (FIG. 2C, panel 6). Beside the flat ribbon-like fibrils in the same preparation, single filaments (FIG. 2C, panel 3) and fibrils composed of two filaments ‘unzipped’ at the edge (FIG. 2C, panels 4, 5) were found.

PK-resistance has been historically used to distinguish PrP^{C} from PrP^{Sc}. Treatment of PrP^{Sc} with PK generates a PK-resistant core encompassing residues ˜90-231, referred to as PrP 27-30. Therefore, it was determined whether any of the two abnormal isoforms generated in vitro have a similar PK-resistant core. Upon incubation at PK/rPrP ratios 1:10,000, 1:5,000, 1:1,000, and 1:500 both the β-oligomer and the amyloid form retained a substantial fraction of intact full-length polypeptide (23 kDa band) and displayed several partially resistant fragments with molecular weights in the range of 16 to 21 kDa (FIG. 2D, panels 1 and 2). However, upon increasing the PK/rPrP ratio to 1:100 and 1:50, only the amyloid form showed a PK-resistant band with molecular mass of 16 kDa, which was totally absent in the β-oligomeric form. The PK-resistant fragment of 16 kDa contained epitopes to Fabs P (residues 96-105, panel 2) and to Fabs R1 (residues 225-231, panel 3) and had an SDS-PAGE mobility similar to that of rPrP 89-230. To confirm that the N-terminal region of rPrP was digested in the amyloid form, antibodies specific to an epitope encompassing residues 79-97 (Ab 79-97) were used. It was found that the partially resistant fragments with molecular weights of ˜16-21 kDa are immunoreactive to Ab 79-97, but only when the amyloid fibrils are treated with low concentrations of PK. Upon treatment with high concentrations of PK all of these fragments disappeared, showing only trace amounts of the 16 kDa band (FIG. 2D, panel 4). Taken together this data indicate that PK-cleavage sites are located within the epitope 79-97, which is cleaved off as the concentration of PK increases.

The results shown herein confirm that the structural transition of rPrP from the native conformation to abnormal isoforms is characterized by an increase in the amount of β-sheet structures, enhanced resistance to PK digestion, and by polymerization into either spherical particles or fibrils. The results also show that the two abnormal isoforms are conformationally different and that only the amyloid form has physical properties similar to that of PrP^{Sc}.

In Vitro Conversion into the Amyloid Form Mimics a Transmission Barrier.

Autocatalytic conversion from PrP^{C} into PrP^{Sc} is believed to be a key feature that underlies the molecular basis of the transmissible form of prion diseases (1; 35). An autocatalytic mechanism of prion replication displays strong species specificity with respect to amino acid sequences of the two interacting isoforms, PrP^{C} and PrP^{Sc}, known as a transmission barrier. The transmission barrier manifests itself as a prolongation of the incubation time when the sequence of PrP^{Sc} in the inoculum does not match that of PrP^{C} in the recipient animals (36). In particular, the transmission barrier was observed when full length PrP^{Sc} was inoculated into transgenic mice expressing PrP composed of 106 amino acid residues (37). Therefore, it was decided to determine whether in vitro conversion of rPrP into the amyloid form displays a similar transmission barrier.

By looking at the kinetics of amyloid formation, it was found that manual withdrawing of aliquots for the ThT assay and other factors related to manual manipulations have a profound effect on the reproducibility of the kinetics. To reduce error from manual handling of individual samples the manual assay format was changed to an automated one using 96-well plates (see Materials and Methods). The indisputable advantage of the new format was the ability to monitor the conversion for a long period of time without manual intervention. Furthermore, as ThT fluorescence was monitored directly from 96-well plate without withdrawing aliquots, the concentration of rPrP in the conversion reaction carried out in the automated format was substantially reduced. Advantageously, the inclusion of ThT did not alter the dynamics of the conversion pattern.

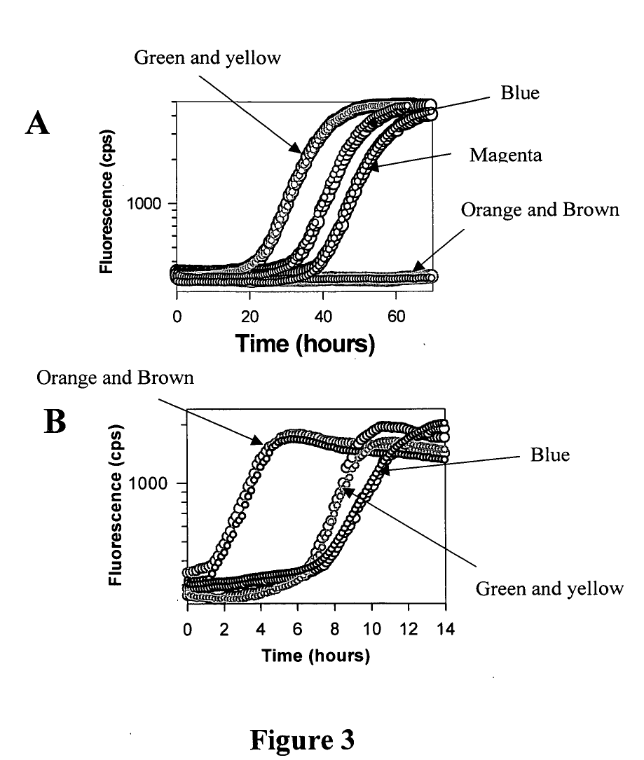

To investigate the transmission barrier, the conversion of rPrP 106 was seeded with preformed amyloid of either rPrP 106 or full-length rPrP (FIG. 3). Under the experimental conditions employed, spontaneous conversion of rPrP 106 did not occur even after 70 h of incubation (FIG. 3A). However, seeding with 2% and 0.2% of preformed fibrils of rPrP 106 induced the conversion reaction with a lag-phase of 20 h and 30 h, correspondingly. On the other hand, seeding with 2% of the preformed fibrils of full-length rPrP was less efficient than seeding with 0.2% that of rPrP 106. This result was consistent with observation that transmission of PrP^{Sc} 106 prions in transgenic PrP 106 mice induced disease after only ˜66 days, while full length PrP^{Sc} produced disease after ˜300 days in these mice (37).

The transmission barrier was even more evident when they inoculated PrP^{Sc} 106 prions into transgenic mice expressing full length PrP^{C}. These mice normally develop disease ˜50 days after inoculation with the RML strain of PrP^{Sc}. However, they did not show any signs of prion disease after inoculation with PrP^{Sc} 106 (37). Similarly, it was found that only preformed fibrils of full-length rPrP, but not those of rPrP 106, were capable of seeding the conversion of full-length rPrP (FIG. 3B). This result illustrates that in vitro conversion into the amyloid form mimics the transmission barrier and that templating with seeds matching the substrate is critical for efficient conversion.

β-Structure of the Amyloid Fibrils is Resistant to Thermal Denaturation and PK-Digestion.

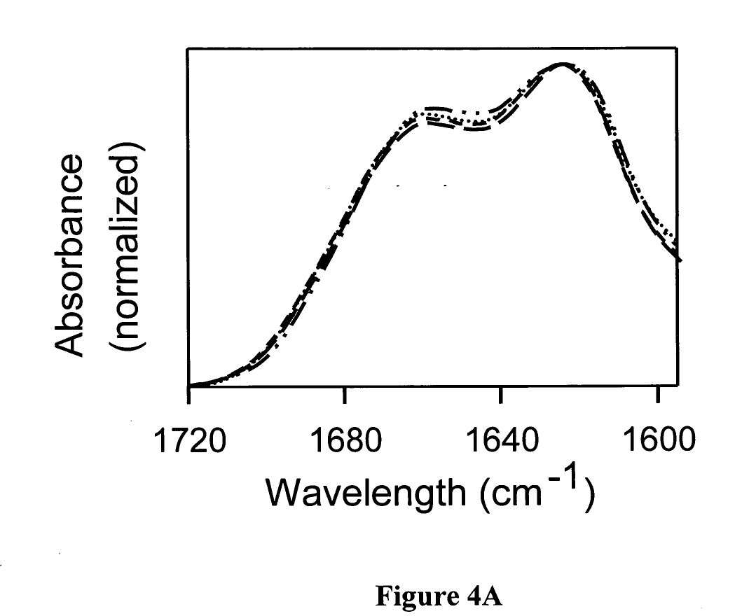

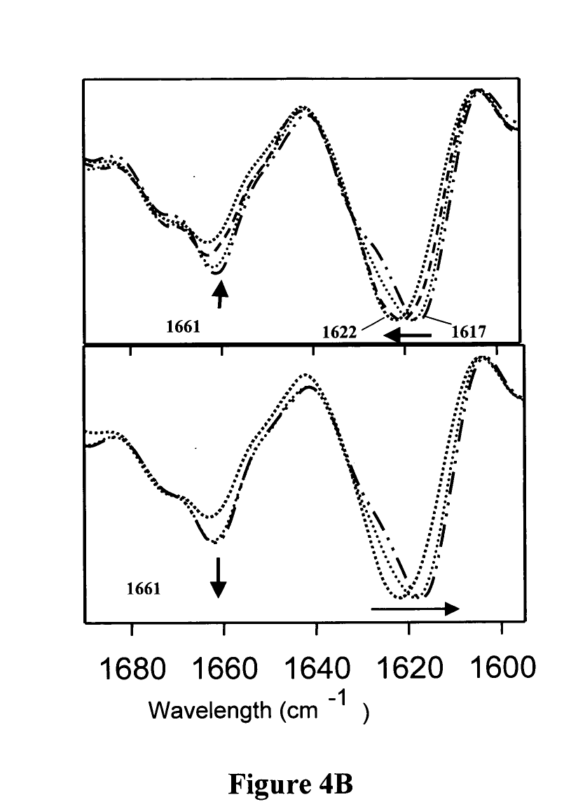

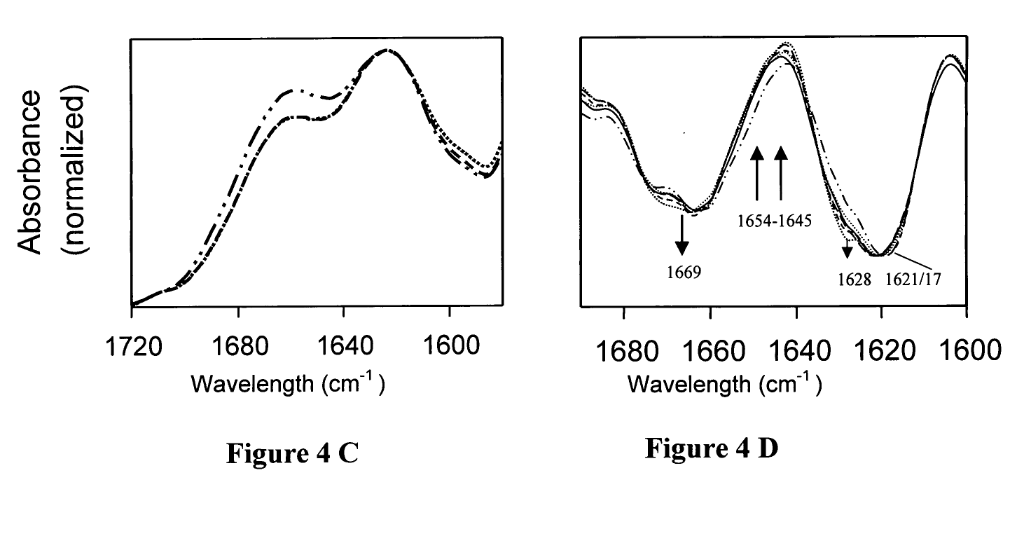

PrP^{Sc} is known to exhibit extremely high conformational stability towards thermal deactivation. To test whether the amyloid form of rPrP possesses increased thermodynamic stability FTIR spectroscopy was employed and spectra was recorded at temperatures between 20° C. and 80° C. using a BioATR cell, which allows FTIR spectra to be collected from aqueous solution (FIG. 4 A,B). An increase of temperature from 20° C. to 80° C. was accompanied by the gradual shift of an absorbance band centered at 1617 cm⁻¹ to 1621 cm⁻¹, while the relative intensity of this band remained stable (FIG. 4 B, top panel). This result indicates that β-sheet structures with strong intermolecular hydrogen bonds were preserved but acquired greater dynamic flexibility at higher temperatures. In parallel, a gradual decrease of an absorbance band at 1661 cm⁻¹ was observed, which corresponds to unfolding of loops, turns and α-helical structures. Both changes, the shift of the band at 1617 cm⁻¹ and the melting of band at 1661 cm⁻¹, were reversible, as both bands returned to their original positions after cooling back to 20° C. (FIG. 4 B, bottom panel). These data demonstrate that the amyloid form exhibits remarkable resistance toward thermal denaturation and that β-structures account for such high thermodynamic stability.

Next it was determined the extent to which amyloid secondary structure is affected by digestion with PK. Within the first 20 min of incubation with PK a substantial decrease in the absorbance between 1654 cm⁻¹ and 1645 cm⁻¹ was observed that indicates rapid reduction in α-helical structure and random coil, respectively (FIG. 4 C). On the other hand, the relative intensity of the major band centered at 1617 cm⁻¹, a characteristic of β-sheet structures with strong hydrogen bonds, remained stable for up to four hours of incubation with PK. The second derivative analysis revealed a slight shift of this band from 1617 cm⁻¹ to 1621 cm⁻¹ indicating that the β-structures acquired a certain degree of flexibility during proteolytic treatment, while still maintaining intermolecular hydrogen bonds (FIG. 4 D). In parallel, the appearance of a minor band at 1628 cm⁻¹ was observed, a characteristic of β-structures with more flexibility. Also an increase in intensity at 1669 cm⁻¹ was noticed which is indicative of β-turns and loops (FIG. 4 D). Taken together, FTIR spectra illustrate that the treatment of amyloid fibrils with PK reduced α-helical structures, increased the amount of β-turns, and preserved β-rich structures, which acquired some conformational flexibility. Therefore, it was decided to determine whether the PK-resistant β-sheet rich core would remain assembled into fibrils.

Limited PK-Digestion Induces Aggregation of the Amyloid Fibrils.

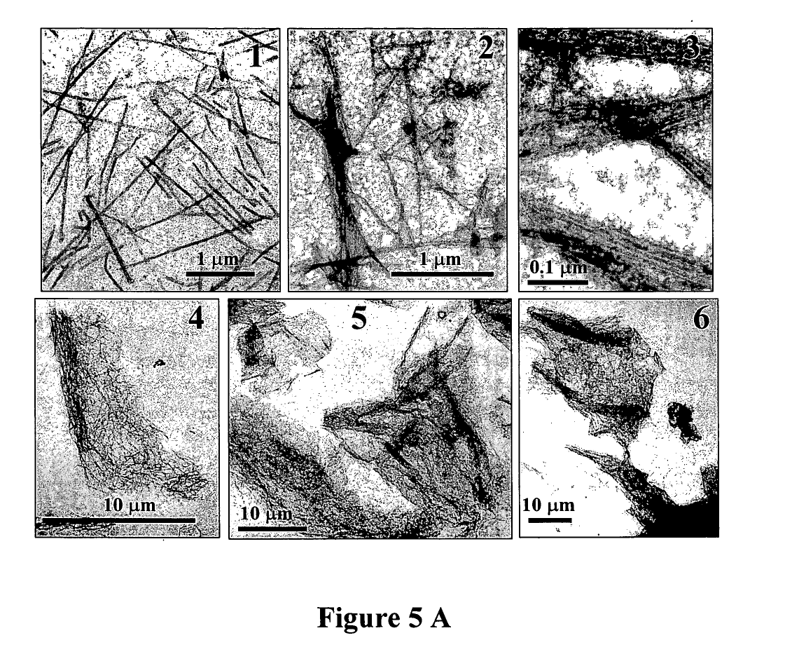

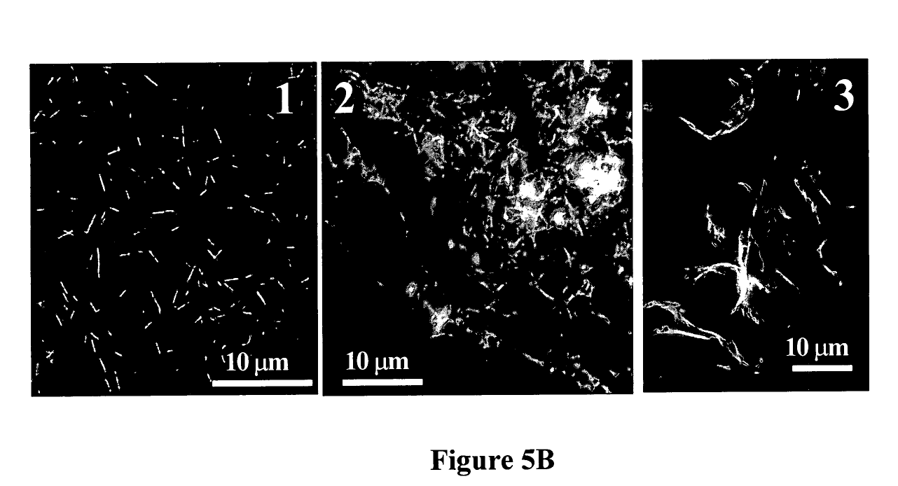

Treatment with PK at PK/rPrP ration of 1:500 did not destroy fibrillar structure, but induced co-aggregation of fibrils (FIG. 5). Using electron microscopy it was found that fibrils laterally attach to each other (FIG. 5A, panels 2, 3) followed by aggregation into larger clumps of various sizes (FIG. 5A, panels 4-6). Because preparation of the sample for electron microscopy includes drying that may cause artifacts in fibril behavior, an alternative technique was developed that allows monitoring the aggregation of fibrils in ‘real time’ using epifluorescent microscopy. Upon placing a drop of solution with amyloid fibrils on a cover slip, fibrils quickly attach to the glass surface and can be easily visualized using an inverted microscope (FIG. 5B, panel 1). Remarkably, injection of PK into the drop induced rapid detachment of fibrils from the surface and co-aggregation in solution (FIG. 5B, panels 2,3). Aggregation occurred within 5-30 min and could be observed in ‘real-time’. Interestingly, PK-treated fibrils retained high ThT-binding capacity illustrating that the PK-resistant fibrillar core maintained an amyloid-specific conformation. At late stages of aggregation the morphology of fibrillar aggregates was similar to that observed by electron microscopy confirming that fibrils treated with PK had an increased tendency for lateral aggregation (FIG. 5B, panel 3).

Harsh PK-Digestion Leads to Fragmentation of the Amyloid Fibrils.

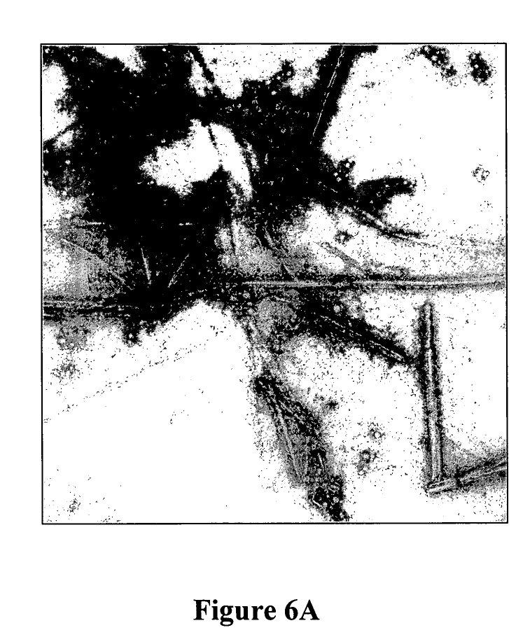

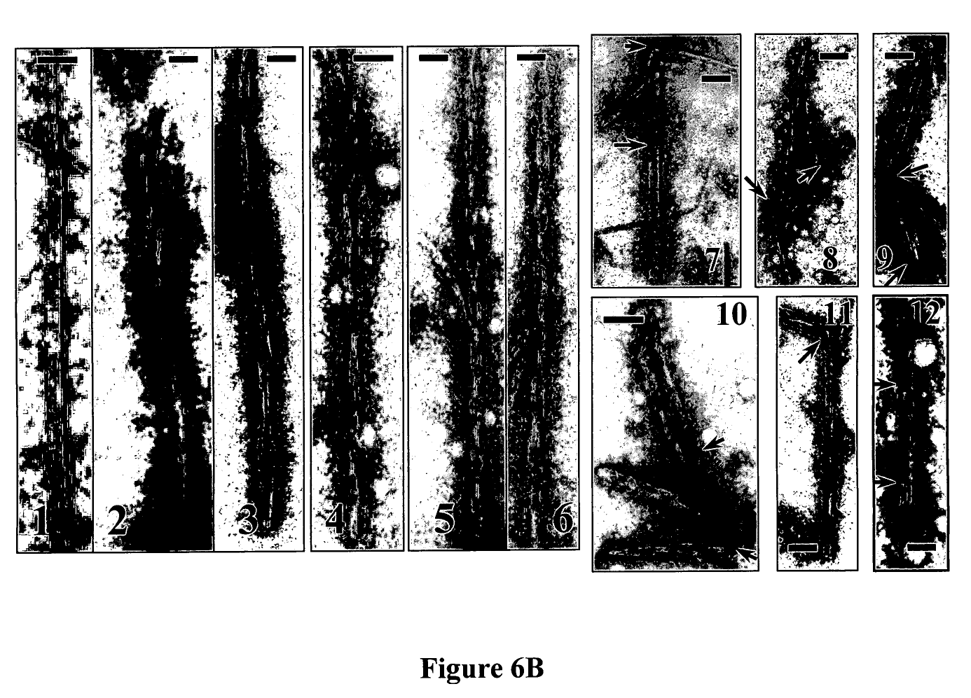

In parallel with aggregation it was noticed that a substantial fraction of the ribbons composed of two non-twisted filaments assembled into thick fibrils (FIG. 6A). The assembly into thick fibrils was especially profound upon treatment with high concentrations of PK (PK/rPrP ratio 1:50). As judged from electron microscopy, the thick fibrils were composed of 2 or more ribbons (FIG. 6B, panels 2-6). These fibrils displayed both untwisted assemblies and assemblies with helical twists. Individual twisted fibrils differed in their helical periodicity and degree of twist (FIG. 6A, panels 5, 6). Interestingly, many fibrils composed of assembled ribbons were untwisted or ‘unzipped’ along the edges (FIG. 6A, panels 2-4). A few fibrils also showed single filaments ‘unzipped’ from fibrils (FIG. 6A, panel 5).

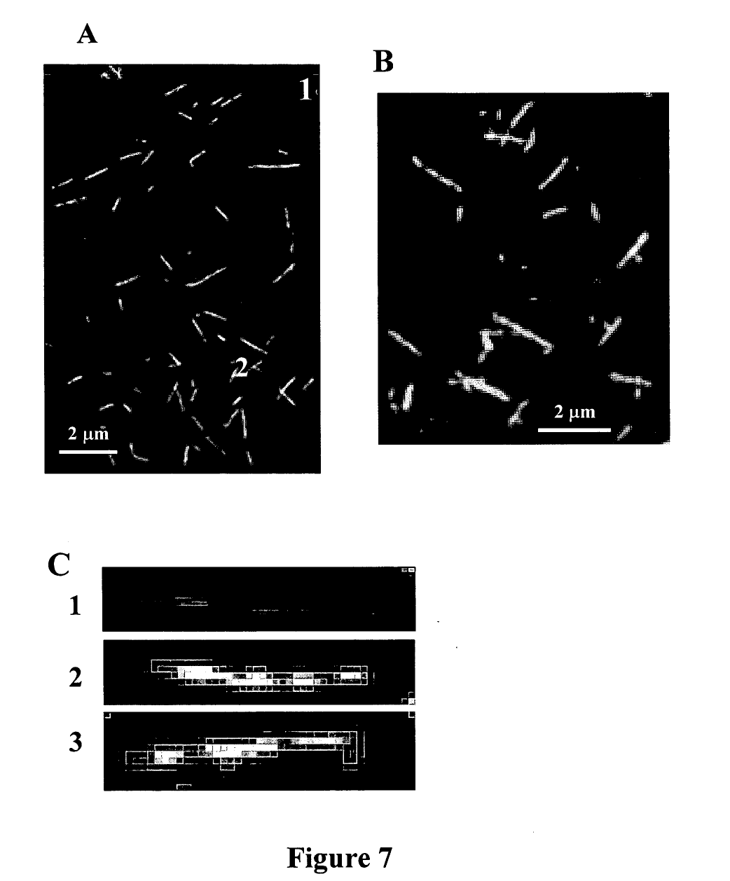

Dramatic differences in morphology of intact and PK-treated fibrils were also seen using epifluorescent microscopy imaging. Fibrils treated with PK were significantly brighter than untreated fibrils (FIG. 7 A, B). Furthermore, imaging of single fibrils revealed that PK-treated fibrils have an alternating pattern of bright and dim emission distributed along the Z-axis, while untreated fibrils displayed a ThT-emission that was uniform along the Z-axis (FIG. 7C, panels 1-3). Differences in brightness and emission patterns between untreated and PK-treated fibrils were consistent with the results obtained by electron microscopy and indicated that the fibrils formed upon incubation with PK have a more complex ultrastructure.

It is noteworthy that unlike intact untreated fibrils (FIG. 6B, panel 1), the PK-treated fibrils had a very high tendency ‘to bend’ and to fragment into shorter pieces (FIG. 6B, panels 7-12). As judged from electron microscopy, ribbons not treated with PK had a linear shape, displayed no ‘bending’ and only seldom had fragmentations (FIG. 5A, panel 1, FIG. 6B, panel 1). However, upon incubation with PK fibrils displayed numerous bends and fragmentations (FIG. 6A). Detailed examination revealed that the fragmentations could occur either across a whole cross-section of fibrils or only across a single ribbon (FIG. 6B, panels 7-12). Taken together, our studies illustrate that the PK-resistant fragments of the amyloid form remained assembled in the fibrillar form and maintained β-sheet rich amyloid specific structure.

PK-Treated Fibrils Preserve High Seeding Activity.

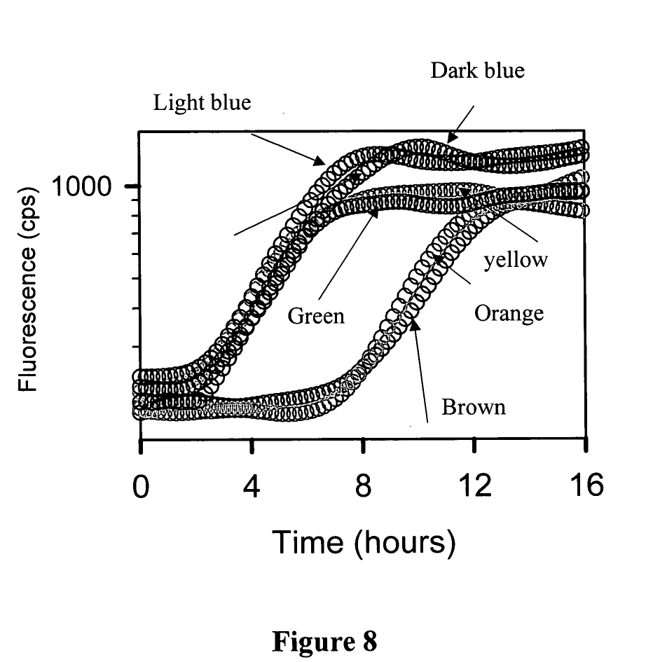

Despite the lack of an N-terminal region, PrP 27-30 is known to preserve a high titer of infectivity. Therefore it was decided to test whether proteolytic digestion of the amyloid form affects its self-propagating activity. FIG. 8 demonstrates that seeding of the in vitro reaction with fibrils not treated with PK substantially reduced the lag-phase (FIG. 8). Remarkably, the fibrils pretreated with PK (PK/rPrP ratio of 1:50) for 1 h at 37° C. showed seeding activities similar to that displayed by untreated fibrils as judged from the length of the lag-phase. In the experimental conditions employed for PK-digestion, no rPrP molecules remained intact as judged from western blotting (FIG. 2D, panel 2). This experiment demonstrates that despite the cleavage of the N-terminal region, PK-treated fibrils possess high seeding activity in a cell-free conversion system.

Conversion of the prion protein from the cellular to pathological isoform plays a central role in prion disorders. It was demonstrated herein that full-length rPrP is able to form several structurally distinct non-native isoforms in the absence of a cellular environment or PrP^{Sc}-template. Under acidic pH, rPrP converts into the β-oligomer, while under neutral and slightly acidic pH we observed formation of amyloid fibrils (FIG. 1 C, D). The results of these experiments are consistent with a model proposed earlier for the conversion of rPrP 90-231 (30; 31). This model postulates that α-rPrP exists in a slow equilibrium with the β-oligomer, which is shifted toward the β-oligomer at acidic pH, but favors α-rPrP at neutral pH (7). The β-oligomer is not on the kinetic pathway to the amyloid form, which is formed at neutral or slightly acidic pH (FIG. 9).

At the same time, the current study raises a question of whether any of the abnormal isoforms that can be generated in vitro are also produced in cells. It was found that the conversion into the β-oligomer occurs predominantly at acidic pH and at high protein concentrations. Therefore, efficient assembly into the oligomeric form in cells would require non-physiological concentrations of PrP and abnormally low pH. In contrast, the amyloid fibrils are formed at physiological pH values and at much lower protein concentrations. Even at 1.0 uM the critical concentration of rPrP was not reached and required for triggering the amyloid formation. The concentrations of rPrP required to produce the amyloid in vitro are similar to those found in normal brains (38). This result demonstrates that full-length PrP exhibits a high propensity to form amyloid fibrils. Why then, despite this high amyloidogenic propensity, does the process of spontaneous conversion of PrP^{C} into PrP^{Sc} occur only rarely in vivo? Interestingly, the conversion reaction is characterized by longer lag-phase and lower yield (32). Taken together the data shown herein relating to in vitro conversion is consistent with the proposition that under normal physiological conditions spontaneous conversion of PrP^{C} into PrP^{Sc} is extremely inefficient, providing an explanation for the very low occurrences of sporadic Creutzfeldt-Jakob Disease (39).

In contrast to the process of conversion to the β-oligomer, the kinetics of fibril formation display attributes of autocatalytic mechanism, such as lag-phase and seeding phenomena According to the template-assisted model, beside having a catalytic role, PrP^{Sc} acts as a template providing conformational constraints for the conversion of PrP^{C} into nascent PrP^{Sc} (40; 41). Templating and catalytic roles of PrP^{Sc} are closely related to each other. When the sequence of PrP^{Sc} does not match that of PrP^{C}, a transmission barrier is observed (36). This transmission barrier can be attributed to the low catalytic efficacy of PrP^{Sc} to propagate its pathological conformation due to the miss-match between amino acid sequences of the template and of the substrate. In the current study, using full-length rPrP and rPrP 106, it was demonstrated that in vitro conversion exhibits high selectivity of seeding and recapitulates a transmission barrier observed in vivo. Thus, fibrils of full-length rPrP were able to seed the conversion of rPrP 106 although such cross-seeding was of low efficiency. On the other hand, fibrils of rPrP 106 did not show any seeding activity toward the full-length rPrP. This result is in accordance with the original observation that mice expressing PrP 106 were susceptible to full-length PrP^{Sc}, but they developed the disease only after a prolonged incubation period (37). However, mice expressing full-length PrP^{C} were resistant to PrP^{Sc} 106. Strong selectivity in cross-seeding suggests that the amyloid forms generated in vitro act not only as catalytic centers but also as templates.

Cell-free conversion of full-length mammalian rPrP into amyloid conformation has never been achieved before. Formation of β-sheet rich species referred to as amyloidogenic unfolding intermediates was previously reported for sheep rPrP variants (42). These β-sheet rich species were capable of binding ThT, although their binding capacity was similar to that of the β-oligomer reported in the current study and substantially lower than ThT-binding of amyloid forms (FIG. 2A). The present results demonstrates that the amyloid form of the full-length rPrP generated in vitro possesses the basic physical properties of PrP^{Sc}. Thus, the amyloid form shows remarkable stability toward temperature-induced denaturation. Analyses of FTIR spectra revealed that β-structures account for such extreme stability (FIG. 4). Prolonged treatment with PK caused only minor conformational perturbations in the β-sheet rich core and did not destroy the fibrillar assembly. Remarkably, the amyloid fibrils had a C-terminal PK-resistant core similar to that of PrP^{Sc} (13), indicating that the amyloid form and PrP^{Sc} may have similar substructures (FIG. 2D). While it has yet to be determined whether the amyloid form of full-length rPrP is capable of inducing prion disease in experimental animals, the amyloid form faithfully recapitulates the basic physical properties of PrP^{Sc}.

Recent studies indicated that the development of prion disease is modulated by the fine balance between two processes, the autocatalytic propagation versus clearance of PrP^{Sc} (43; 44). Because proteolytic degradation by endogenous proteases is believed to play a role in the clearance of PrP^{Sc}, testing the possible effects that treatment with PK may have on amyloid aggregates was of interest. It was found that upon incubation with PK the N-terminal region was gradually digested (FIG. 2B), while the C-terminal region preserved β-rich fibrillar structure (FIGS. 4-6). These data argue that the N-terminal region is not involved directly in the formation of the fibrillar core. On the other hand, removal of the N-terminal regions resulted in dramatic effects. At PK/rPrP ratio of 1:500 the ribbon-like fibrils underwent lateral co-aggregation followed by formation of large clumps. At the PK/rPrP ratio 1:50 substantial fractions of ribbons self-assembled into fibrils composed of 2 or more ribbons (FIG. 6). Both lateral aggregation and self-assembly can be attributed to the exposure of hydrophobic surfaces as a result of the cleavage of the N-terminal residues. Self-assembly of ribbons into fibrils seem to be facilitated as the PK-digestion progressed, further eliminating electrostatic and steric repulsions of N-terminal regions of neighboring polypeptides. Although N-terminal region does not contribute directly to the structural stability of the fibrils, its cleavage dramatically increases fragility of the fibrils and their ability to bend and break into short pieces (FIG. 6).

Both aggregation of the fibrils and their fragmentation may have physiological implications for development of prion disease (FIG. 9). Thus, aggregation of PrP^{Sc} in the brain into large prion plaques may interfere with the normal function of neurons. On the other hand, fibril fragmentation creates new centers for propagation and, therefore, accelerates the rate of prion replication. Remarkably, fibrils treated with PK did not lose the ability to seed the conversion reactions in fresh reaction mixtures. Interestingly, recent studies by Telling and co-authors demonstrated that inhibitors of cellular Ca²⁺-dependent proteases reduced the rate of PrP^{Sc} accumulation in cultured cells pointing out a potential role of proteolytic enzymes in stimulating prion propagation (44). Taken together the results shown herein are consistent with the hypothesis that the endoproteolytic processing of PrP^{Sc} that occurs in vivo, in parallel with generation of nascent PrP^{Sc}, modulates development of prion diseases (44). As proteolytic digestion may accelerate both clearance and propagation of prions, potential therapeutic strategies that stimulate cellular proteases should be considered with great caution (45).

Presented studies demonstrate that amyloid isoforms biochemically identical to PrP^{Sc} can be generated in vitro in the absence of a cellular environment or PrP^{Sc}-templating. As judged from proteinase K digestion, electron microscopy, Fourier transform infrared spectroscopy (FTIR), and real time fluorescent microscopy, the amyloid form displays physical properties similar to that of PrP^{Sc}. As only miniscule amount of recombinant PrP is sufficient for the reaction, this novel in vitro conversion system should be of great benefit for further studies of the biophysical mechanism of prion propagation.

Abbreviations used: The abbreviations used: PrP, prion protein; rPrP, recombinant full-length PrP; α-rPrP, α-helical isoform of rPrP; rPrP 106, recombinant PrP of 106 residues (deletions are Δ23-88 and Δ141-176); PrP^{C}, cellular isoform of the prion protein; PrP^{Sc}, disease associated isoform of the prion protein; ThT, Thioflavin T; PK, proteinase K; GdnHCl, guanidine hydrochloride; FTIR, Fourier transform infrared spectroscopy; CD, circular dichroism.

### REFERENCE LIST

All reference cited herein are hereby incorporated by reference herein for all purposes.
1. Prusiner, S. B. (1997). Prion diseases and the BSE crisis. Science 278, 245-251.
2. Pan, K.-M., Baldwin, M., Nguyen, J., Gasset, M., Serban, A., Groth, D., Mehlhom, I., Huang, Z., Fletterick, R. J., Cohen, F. E., & Prusiner, S. B. (1993). Conversion of a-helices into b-sheets features in the formation of the scrapie prion proteins. Proc. Natl. Acad. Sci. USA 90, 10962-10966.
3. Caughey, B. W., Dong, A., Bhat, K. S., Ernst, D., Hayes, S. F., & Caughey, W. S. (1991). Secondary structure analysis of the scrapie-associated protein PrP 27-30 in water by infrared spectroscopy. Biochemistry 30, 7672-7680.
4. Prusiner, S. B. (1982). Novel proteinaceous infectious particles cause scrapie. Science 216, 136-144.
5. Hill, A. F., Antoniou, M., & Collinge, J. (1999). Protease-resistant prion protein produced in vitro lacks detectable infectivity. J. Gen. Virol. 80, 11-14.
6. Kirby, L., Birkett, C. R., Rudyk, H., Gilbert, I. H., & Hope, J. (2003). In vitro cell-free conversion of bacterial recombinant PrP to PrPres as a model for conversion. J. Gen. Virol. 84, 1013-1020.
7. Baskakov, I. V., Legname, G., Prusiner, S. B., & Cohen, F. E. (2001). Folding of prion protein to its native a-helical conformation is under kinetic control. J. Biol. Chem. 276, 19687-19690.
8. Swietnicki, W., Morillas, M., Chen, S. G., Gambetti, P., & Surewicz, W. K. (2000). Aggregation and fibrillization of the recombinant human prion protein huPrP90-231. Biochemistry 39, 424-431.
9. Zhang, H., Stockel, J., Mehlhorn, I., Groth, D., Baldwin, M. A., Prusiner, S. B., James, T. L., & Cohen, F. E. (1997). Physical studies of conformational plasticity in a recombinant prion protein. Biochemistry 36, 3543-3553.
10. Jackson, G. S., Hosszu, L. L. P., Power, A., Hill, A. F., Kenney, J., Saibil, H., Craven, C. J., Waltho, J. P., Clarke, A. R., & Collinge, J. (1999). Reversible conversion of monomeric human prion protein between native and fibrilogenic conformations. Science 283, 1935-1937.
11. Sokolowski, F., Modler, A. J., Masuch, R., Zirwer, D., Baier, M., Lutsch, G., M. D. A., Gast, K., & Naumann, D. (2003). Formation of critical oligomers is a key event during conformational transition of recombinant syrian hamster prion protein. J. Biol. Chem. Epub ahead of print.
12. Kazlauskaite, J., Sanghera, N., Sylvester, I., Venien-Bryan, C., & Pinheiro, T. J. (2003). Structural changes of the prion protein in lipid membranes leading to aggregation and fibrillization. Biochemistry 42, 3295-3304.
13. McKinley, M. P., Bolton, D. C., & Prusiner, S. B. (1983). A protease-resistant protein is a structural component of the scrapie prion. Cell 35, 57-62.
14. Prusiner, S. B., McKinley, M. P., Bowman, K. A., Bolton, D. C., Bendheim, P. E., Groth, D. F., & Glenner, G. G. (1983). Scrapie prions aggregate to form amyloid-like birefringent rods. Cell 35, 349-358.
15. Fischer, M., Rülicke, T., Raeber, A., Sailer, A., Moser, M., Oesch, B., Brandner, S., Aguzzi, A., & Weissmann, C. (1996). Prion protein (PrP) with amino-proximal deletions restoring susceptibility of PrP knockout mice to scrapie. EMBO J. 15, 1255-1264.
16. Aronoff-Spencer, E., Burns, C. S., Avdievich, N. I., Gerfen, G. J., Peisach, J., Antholine, W. E., Ball, H. L., Cohen, F. E., Prusiner, S. B., & Millhauser, G. L. (2000). Identification of the Cu²⁺ binding sites in the N-terminal domain of the prion protein by EPR and CD spectroscopy. Biochemistry 39, 13760-13771.
17. Viles, J. H., Cohen, F. E., Prusiner, S. B., Goodin, D. B., Wright, P. E., & Dyson, H. J. (1999). Copper binding to the prion protein: structural implications of four identical cooperative binding sites. Proc. Natl. Acad. Sci. USA 96, 2042-2047.
18. Riek, R., Homemann, S., Wider, G., Glockshuber, R., & Wüthrich, K. (1997). NMR characterization of the full-length recombinant murine prion protein, mPrP(23-231). FEBS Lett 413, 282-288.
19. Donne, D. G., Viles, J. H., Groth, D., Mehlhom, I., James, T. L., Cohen, F. E., Prusiner, S. B., Wright, P. E., & Dyson, H. J. (1997). Structure of the recombinant full-length hamster prion protein PrP(29-231): the N terminus is highly flexible. Proc. Natl. Acad. Sci. USA 94, 13452-13457.
20. Burns, C. S., Aronoff-Spencer, E., Legname, G., Prusiner, S. B., Antholine, W. E., Gerfen, G. J., Peisach, J., & Millhauser, G. L. (2003). Copper Coordination in the Full-Length, Recombinant Prion Protein. Biochemistry 42, 6794-6803.
21. Morante, S., Gonzalez-Iglesias, R., Potrich, C., Meneghini, C., Meyer-Klaucke, W., Menestrina, G., & Gasset, M. (2004). Inter- and intra-octarepeat Cu(II) site geometries in the Prion Protein: Implications in Cu(II) binding cooperativity and Cu(II)-mediated assemblies. J. Biol. Chem. 279, 11753-11759.
22. Jackson, G. S., Murray, I., Hosszu, L., Gibbs, N., Waltho, J. P., Clarke, A. R., & Collinge, J. (2001). Location and properties of metal-binding sites on the human prion protein. Proc. Acad. Natl. Sci. U.S.A. 98, 8531-8535.
23. Gabus, C., Derrington, E., Leblanc, P., Chnaiderman, J., Dormont, D., Swietnicki, W., Morillas, M., Surewicz, W. K., Marc, D., Nandi, P., & Darlix, J. L. (2004). The prion protein has RNA binding chaperoning properties characteristic of nucleocasid protein NCP7 of HIV-1. J. Biol. Chem. 276, 19301-19309.
24. Warner, R. G., Hundt, C., Weiss, S., & Tumbull, J. E. (2002). Identification of the heparan sulfate binding sites in the cellular prion protein. J. Biol. Chem. 277, 18421-18430.
25. Gonzalez-Iglesias, R., Pajares, M. A., Espinosa, C. O. J. C., Oesch, B., & Gasset, M. (2002). Prion Protein Interaction with Glycosaminoglycan Occurs with the Formation of Oligomeric Complexes Stabilized by Cu(II) Bridges. J. Mol. Biol. 319, 527-540.
26. Wong, C., Xiong, L. W., Horiuchi, M., Raymond, L., Wehrly, K., Chesebro, B., & Caughey, B. (2001). Sulfated glycans and elevated temperature stimulate PrP(Sc)-dependent cell-free formation of protease-resistant prion protein. EMBO J. 20, 377-386.
27. Deleault, N. R., Lucassen, R. W., & Supattapone, S. (2003). RNA molecules stimulate prion protein conversion. Nature 425, 717-720.
28. Adler, V., Zeiler, B., Kryukov, V., Kascsak, R., Rubenstein, R., & Grossman, A. (2003). Small, Highly Structured RNAs Participate in the Conversion of Human Recombinant PrPsen to PrPres in Vitro. J. Mol. Biol. 332, 47-57.
29. Wadsworth, J. D. F., Hill, A. F., Joiner, S., Jackson, G. S., Clarke, A. R., & Collinge, J. (1999). Strain-specific prion-protein conformation determined by metal ions. Nat. Cell Biol. 1, 55-59.
30. Baskakov, I. V., Legname, G., Baldwin, M. A., Prusiner, S. B., & Cohen, F. E. (2002). Pathway Complexity of Prion Protein Assembly into Amyloid. J Biol. Chem. 277, 21140-21148.
31. Baskakov, I. V. (2004). Autocatalytic conversion of Recombinant Prion Proteins Displays a Species Barrier. J Biol. Chem. 279, 586-595.
32. Baskakov, I. V., Legname, G., Gryczynski, Z., & Prusiner, S. B. (2004). The peculiar nature of unfolding of human prion protein. Protein Sci. 13, 586-595.
33. Gasset, M., Baldwin, M. A., Fletterick, R. J., & Prusiner, S. B. (1993). Perturbation of the secondary structure of the scrapie prion protein under conditions that alter infectivity. Proc. Natl. Acad. Sci. USA 90, 1-5.
34. Caughey, B., Raymond, G. J., & Bessen, R. A. (1998). Strain-dependent differences in b-sheet conformations of abnormal prion protein. J. Biol. Chem. 273, 32230-32235.
35. Gajdusek, D. C. (1988). Transmissible and non-transmissible amyloidoses: autocatalytic post-translational conversion of host precursor proteins to β-pleated sheet configurations. J. Neuroimmunol. 20, 95-110.
36. Pattison, I. H. & Jones, K. M. (1968). Modification of a strain of mouse-adapted scrapie by passage through rats. Res. Vet. Sci. 9, 408-410.
37. Supattapone, S., Bosque, P., Muramoto, T., Wille, H., Aagaard, C., Peretz, D., Nguyen, H.-O. B., Heinrich, C., Torchia, M., Safar, J., Cohen, F. E., DeArmond, S. J., Prusiner, S. B., & Scott, M. (1999). Prion protein of 106 residues creates an artificial transmission barrier for prion replication in transgenic mice. Cell 96, 869-878.
38. Safar, J., Wille, H., Itri, V., Groth, D., Serban, H., Torchia, M., Cohen, F. E., & Prusiner, S. B. (1998). Eight prion strains have PrP^{Sc} molecules with different conformations. Nat. Med. 4, 1157-1165.
39. Masters, C. L., Harris, J. O., Gajdusek, D. C., Gibbs, C. J., Jr., Bernouilli, C., & Asher, D. M. (1978). Creutzfeldt-Jakob disease: patterns of worldwide occurrence and the significance of familial and sporadic clustering. Ann. Neurol. 5, 177-188.
40. Cohen, F. E., Pan, K.-M., Huang, Z., Baldwin, M., Fletterick, R. J., & Prusiner, S. B. (1994). Structural clues to prion replication. Science 264, 530-531.
41. Cohen, F. E. & Prusiner, S. B. (1998). Pathologic conformations of prion proteins. Annu. Rev. Biochem. 67, 793-819.
42. Rezaei, H., Choiset, Y., Eghiaian, F., Treguer, E., Mentre, P., Debey, P., Grosclaude, J., & Haertle, T. (2002). Amyloidogenic Unfolding Intermediates Differentiate Sheep Prion Protein Variants. J. Mol. Biol. 322, 799-814.
43. Peretz, D., Williamson, R. A., Kaneko, K., Vergara, J., Leclerc, E., Schmitt-Ulms, G., Mehlhorn, I. R., Legname, G., Wormald, M. R., Rudd, P. M., Dwek, R. A., Burton, D. R., & Prusiner, S. B. (2001). Antibodies inhibit prion propagation and clear cell cultures of prion infectivity. Nature 412, 739-743.
44. Yadavalli, R., Guttmann, R. P., Seward, T., Centers, A. P., Williamson, R. A., & Telling, G. C. (2004). Calpain-dependent endoproteolytic cleavage of PrP^{Sc} modulates scrapie prion. J. Biol. Chem. 279, 21948-21956.
45. Checler, F. & Vincent, B. (2002). Alzheimer's and prion diseases: distinct pathologies, common proteolytic denominators. Trends in Neurosciences 25, 616-620.
46. Baskakov, I. V., Aagaard, C., Mehlhom, I., Wille, H., Groth, D., Baldwin, M. A., Prusiner, S. B., & Cohen, F. E. (2000). Self-assembly of recombinant prion protein of 106 residues. Biochemistry 39, 2792-2804.
47. Shinagawa, M., Munekata, E., Doi, S., Takahashi, K., Goto, H., & Sato, G. (1986). Immunoreactivity of a synthetic pentadecapeptide corresponding to the N-terminal region of the scrapie prion protein. J. Gen. Virol. 67, 1745-1750.

That which is claimed is:

1. An in vitro method for converting a prion protein to an amyloid form, the method comprising:
a) providing a conversion solution comprising guanidine hydrochloride (GdnHCl);
b) adding a recombinant full-length prion protein with an intact S-S bind to the conversion solution;
c) maintaining the pH in the solution in a range from about 5.5 to about 7.0;
d) exposing the recombinant prion protein to the solution under essentially continuance shaking for a sufficient time to form an amyloid structure

2. The method of claim 1 wherein the pH is about 6.5.

3. The method according to claim 1, further comprising the addition of urea to the conversion solution.

4. The method according to claim 1, wherein the shaking was at about 400 to 700 RPM.

5. The method according to claim 1, wherein the amyloid structure showed some structure with strong intermolecular hydrogen bonds.

6. An in vitro method for converting a prion protein to a β-oligomer form, the method comprising:
a) providing a conversion solution comprising guanidine hydrochloride (GdnHCl);
b) adding a recombinant full-length prion protein or fragment thereof to the conversion solution;
c) maintaining the pH in the solution in a range from about 3.0 to about 4.0;
d) exposing the recombinant prion proteins to the solution for a sufficient time to form an β-oligomer form.

7. The method of claim 2, wherein the pH is about 3.7.

8. An automated in vitro method of monitoring conversion kinetics of the conversion of a full-length prior protein or fragments thereof, the method comprising:
a) providing a conversion solution comprising guanidine hydrochloride (GdnHCl) and Thioflavin T (ThT);
b) adding a full-length prion protein or fragment thereof to the conversion solution;
c) maintaining the pH in the solution in a range from about 3.0 to about 6.5; and
d) monitoring the conversion kinetics by measuring the fluorescence intensity corresponding to the conversion.

9. The automated method according to claim 8, wherein the pH is from about 5.5 to about 6.5 and the continuously shaking the prion protein solution to form an amyloid form.

10. The automated method according to claim 8, wherein the pH is from about 3.0 to about 4.0 and exposing the prion protein a sufficient time to form a β-oligomer form.

11. An automated in vitro method for determining test compounds that inhibit or reduce the conversion of a full-length prior protein or fragments thereof into an amyloid or β-oligomer, the method comprising:
a) providing a conversion solution comprising guanidine hydrochloride (GdnHCl) and Thioflavin T (ThT);
b) adding a full-length prion protein or fragment thereof to the conversion solution;
c) maintaining the pH in the solution in a range from about 3.0 to about 7.0;
d) introducing the test compound; and
e) monitoring the conversion kinetics relative to a control sample without the test compound by measuring the fluorescence intensity corresponding to the conversion.

12. The automated method according to claim 11, wherein the pH is from about 5.5 to about 6.5 and continuously shaking the prion protein solution to form an amyloid form.

13. The automated method according to claim 11, wherein the pH is from about 3.0 to about 4.0 and exposing the prion protein a sufficient time to form a β-oligomer form.

14. A kit for determining test compounds that inhibit or reduce the conversion of a full-length prior protein or fragments thereof into a β-oligomer or amyloid form, the kit comprising:
a) a conversion solution comprising guanidine hydrochloride (GdnHCl) and Thioflavin T (ThT);
b) a pH altering compound for maintaining the conversion in a range from about 3.0 to about 7.0, wherein a full-length prion protein and test compound are added to the conversion solution and monitoring conditions to determine if the test compound inhibits or reduces conversion.

15. The kit according to claim 14, wherein the solution is maintained at a pH from about 5.5 to about 6.5 and maintained under essentially continuance motion to form the amyloid form.

16. The kit according to claim 14, wherein the solution is maintained at a pH from about 3.0 to about 4.0 to form the β-oligomer form.


SEQUENCE LISTING (2 sequences)

SEQ ID NO: 1 - DNA, 762 bp, Mus musculus
  CDS (1)..(762)
atggcgaaccttggctactggctgctggccctctttgtgactatgtggactgatgtcggcctctgcaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccataatcagtggaacaagcccagcaaaccaaaaaccaacctcaagcatgtggcaggggctgcggcagctggggcagtagtggggggccttggtggctacatgctggggagcgccatgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagcagcaccgtgcttttctcctcccctcctgtcatcctcctcatctccttcctcatcttcctgatcgtggga

SEQ ID NO: 2 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG